(19) United States
(12) Patent Application Publication
(10) Pub. No.: US 2026/0197947 A1
(43) Pub. Date: Jul. 9, 2026

(54) INTEGRATED SYSTEM FOR TEXTILE HYBRID ELECTRONICS VIA LASER HYBRID MANUFACTURING

(71) Applicant: Zhejiang University, Hangzhou (CN)

(72) Inventors: Kaichen Xu, Hangzhou (CN); Huayu Luo, Hangzhou (CN); Chongyi Xu, Hangzhou (CN); Ziguan Jin, Hangzhou (CN); Geng Yang, Hangzhou (CN); Huayong Yang, Hangzhou (CN)

(21) Appl. No.: 19/433,055

(22) Filed: Dec. 25, 2025

(30) Foreign Application Priority Data
Jan. 3, 2025 (CN) 202510010843.8

(51) Int. Cl. H05K 3/341; A61B 5/00; A61B 5/256; A61B 5/282; B23K 1/00; B23K 1/005; B23K 26/359; B23K 26/38; B23K 26/402; B23K 101/42; B23K 103/00; H05K 1/03; H05K 3/10; H05K 3/40

(52) U.S. Cl. CPC H05K 3/341; A61B 5/256; A61B 5/282; A61B 5/6804; B23K 1/0016; B23K 1/0056; B23K 26/359; B23K 26/38; B23K 26/402; H05K 1/038; H05K 3/107; H05K 3/4053; A61B 2562/12; B23K 2101/42; B23K 2103/38; H05K 2201/10151; H05K 2203/107

(57) ABSTRACT

An integrated system for textile hybrid electronics via laser hybrid manufacturing includes surface mounted devices (SMDs), laser-induced flexible sensors, a textile substrate, conductive textile traces and physiological electrodes, and vertical interconnect accesses (VIAs). By leveraging a material modification capability of a laser, patterned sensors can be prepared directly on the textile substrate. A precision cutting capability of a laser is utilized to create patterned flexible electrodes and interconnect wires. Furthermore, highly stable VIAs are formed by infiltrating the fabric's intrinsic structure with conductive composites. By integrating the aforementioned preparation techniques, a double-layer fabric circuit board can be realized on various daily-use and specialized textiles. Subsequently, the SMDs are soldered onto the board to form an integrated system for textile hybrid electronics. The present disclosure achieves heterogeneous integration of rigid electronics, flexible electronics, and textiles into a fully functional, breathable, and wearable system.

### CROSS-REFERENCE TO RELATED APPLICATION

This application claims priority of Chinese Patent Application No. 202510010843.8, filed on Jan. 3, 2025, the entire contents of which are incorporated herein by reference.

### TECHNICAL FIELD

The present disclosure belongs to the technical field of wearable electronics, and relates to an intelligent electronic textile, particularly to an integrated system for textile hybrid electronics via laser hybrid manufacturing.

### BACKGROUND

Textiles are among the most intimate material carriers in human daily life. Textile-based electronic systems can effectively combine the information-processing capabilities of electronic devices with the inherent advantages of textiles, endowing breathable, protective, and skin-friendly fabrics with sensing, actuation, communication, and computational functions, thereby bridging the gap between humans and flexible electronic systems. Such systems hold broad application prospects in fields, such as precision medicine and human-machine interaction.

Based on integration methods of silicon-based electronic components, there are primarily three approaches to realize textile-based electronic systems. The first approach involves integrating conventional silicon-based chips onto commercial substrates separate from the textile, leaving only inherently flexible components on the fabric itself. However, this approach fails to address the mechanical incompatibility between the commercial substrate and the human body surface, making it difficult to ensure wearing comfort. The second approach is to abandon silicon-based chips and employ functionalized fibers woven directly into electronic devices. Such all-fiber electronic textiles demonstrate capabilities in signal processing, visual interaction, and wireless transmission, but face challenges in terms of functional versatility and interface compatibility. The third approach integrates silicon-based chips directly onto the textile substrate. This approach is more straightforward and flexible, fully leveraging the advantages of silicon-based electronics, yet it still requires improvements in functional density, wearability comfort, and attachment stability.

The present disclosure solves the integration problem of combining existing textile-based electronic systems with rigid components. By utilizing laser hybrid manufacturing as a core technique and incorporating process improvements such as transfer printing, the present disclosure creates an integrated heterogeneous system. This system includes functionalized textiles, flexible electronic elements, silicon-based chips, double-layer conductive textile traces, and vertical interconnect accesses (VIAs). Furthermore, the system can achieve universal circuit functions on a single-piece textile, thereby enhancing portability and functional density of the textile-based electronic system.

Compared with existing electronic textile technologies, the present disclosure achieves the formation of independently routed circuits on front and back faces of the textile, thereby significantly enhancing circuit integration density and expanding application versatility. The system is compatible with conventional surface mounted device (SMD) packages, and its conductive traces can be soldered, resulting in wider applicability.

### SUMMARY

To overcome the deficiencies of existing technologies, an objective of the present disclosure is to provide an integrated system for textile hybrid electronics via laser hybrid manufacturing. This system is achieved by utilizing a thermal effect of a carbon dioxide (CO₂) laser or a continuous-wave laser to modify a textile surface, thereby forming laser-induced flexible sensors with sensing functionality. Concurrently, a precision cutting capability of a pulsed laser is employed to create patterned conductive textile traces and physiological electrodes on a textile substrate. Furthermore, monolithic integration of electronic components with textiles is achieved by integrating multiple laser manufacturing technologies with other manufacturing processes. The manufacturing method provided in the present disclosure can achieve the integration of functionalized textiles, flexible electronic elements, rigid electronic components, and double-layer conductive textile traces onto a fabric. The resulting integrated system for textile hybrid electronics not only performs multiple functions such as sensing, detection, and signal transmission, but also exhibits stable performance with resistance to washing and mechanical crumpling.

To solve the above technical problems, the present disclosure employs the following technical solution:
an integrated system for textile hybrid electronics via laser hybrid manufacturing includes a textile substrate, conductive textile traces and physiological electrodes, VIAs, laser-induced flexible sensors, and SMDs, in which all electronic components are densely integrated on a single textile substrate and distributed on surfaces of two sides of the textile substrate, interconnected to achieve complete circuit functionality; and a preparation process specifically includes the steps of:
   S1, formation of the laser-induced flexible sensors: processing the textile substrate or precursor materials on a surface of the textile substrate using a photothermal effect of either a CO₂ laser or a 532 nm continuous-wave green laser according to a pre-designed pattern, and inducing reactions such as carbonization, phase separation, or reduction-sintering, to generate the laser-induced flexible sensors on one side of the textile substrate for detecting physical and chemical signals on a human body surface; and electrically routing the laser-induced flexible sensors out from the other side of the textile substrate;
   S2, formation of the VIAs: employing a pneumatic method to infiltrate conductive paste into textured structures of the textile substrate at locations on the textile substrate where VIAs are required to connect circuits on two sides of the textile substrate, to achieve electrical connectivity between front and back faces at corresponding regions of the textile substrate;
   S3, formation of the conductive textile traces and physiological electrodes: cutting a conductive textile using an ultraviolet (UV) nanosecond laser according to a pre-designed pattern to form the conductive traces and physiological electrodes; and transferring the conductive traces and electrodes onto the two sides of the textile substrate using a transfer-printing process, causing the conductive traces and electrodes on the two sides to be electrically interconnected through the VIAs; and
   S4, integration of the SMDs: soldering the SMDs onto the conductive textile traces using a low-melting solder paste, forming a complete circuit.

Further, the textile substrate is made of one or more materials selected from the group consisting of: modal knitted fabric, medical non-woven fabric, polyimide (PI) fabric, and taffeta woven fabric.

Further, the formation of the laser-induced flexible sensors is specifically achieved as follows: using a CO₂ laser, one side of the textile substrate is carbonized according to a pre-designed pattern, generating a laser-induced graphene (LIG) sensor; or a 532 nm wavelength continuous-wave green laser is employed to induce a phase-separation reaction in poly(3,4-ethylenedioxythiophene):poly(styrene sulfonate) (PEDOT:PSS) on a textile surface, generating a laser-induced PEDOT:PSS sensor; or using the same 532 nm continuous-wave green laser, a reduction-sintering reaction is induced in silver nano-ink on the textile surface, generating a laser-induced metal sensor. All the sensors are routed electrically out from the other side of the textile substrate.

Further, the conductive paste is an intermetallic compound formed from a liquid metal (LM)/copper nanoparticle (Cu NP) mixture, which is prepared using a centrifugal mixer.

Further, the conductive textile traces and physiological electrodes are prepared by combining laser cutting of conductive textiles with a transfer printing process: during laser cutting, a water-soluble sacrificial layer (SL) is utilized to temporarily secure the conductive textile traces and physiological electrodes; after cutting is completed, the conductive textile traces and physiological electrodes are transferred onto the textile substrate; and the SL is dissolved in water to prevent laser-induced damage to the textile substrate.

Further, a method for preparing the conductive textile traces and physiological electrodes specifically includes: laminating an adhesive film (AF) onto one side of a metalized textile (MT) and laminating a water-soluble SL onto the other side of the MT to form an AF/MT/SL laminate; cutting AF and MT layers into patterned conductive circuits using a UV nanosecond laser while keeping the SL intact; adhering the entire laminate to the textile substrate with an AF face facing down; immersing the textile substrate in water to soften the SL, followed by removing the SL; drying the remaining AF/MT layers on the textile substrate; and removing excess AF/MT material outside the patterned conductive circuits to form the conductive textile traces or physiological electrodes.

Further, the low-melting solder paste is a mixed slurry of tin-based solder paste and aluminum flux. The present disclosure has the following beneficial effects.

The system of the present disclosure achieves an independently routed double-layer conductive circuit on a single-piece textile, allowing for high-density circuit layouts. Moreover, wearing comfort is ensured by isolating rigid electronic components on one side and skin-conformable flexible electronic components on the other side. Building upon this foundation, the system achieves acquisition and wireless transmission of multiple physiological signals on the single-piece textile, which has been validated through human motion experiments.

### BRIEF DESCRIPTION OF THE DRAWINGS

FIG. 1 shows a schematic diagram of a manufacturing process for an LIG sensor.

FIG. 2 shows a schematic diagram of a manufacturing process for an LM/Cu NP VIAs.

FIG. 3A shows a resistance change of a single-layer conductive textile trace under repeated bending; and

FIG. 3B shows a resistance change of a double-layer conductive textile trace with four LM/Cu NP VIAs under repeated bending.

FIG. 4A shows an image of LM/Cu NP VIAs manufactured on modal knitted fabric with an illuminated light-emitting diode (LED);

FIG. 4B shows an image of LM/Cu NP VIAs manufactured on medical non-woven fabric with an illuminated LED;

FIG. 4C shows an image of LM/Cu NP VIAs manufactured on PI woven fabric with an illuminated LED; and

FIG. 4D shows an image of LM/Cu NP VIAs manufactured on taffeta woven fabric with an illuminated LED.

FIG. 5 shows a schematic diagram of manufacturing for the double-layer conductive textile trace.

FIG. 6 shows a schematic diagram of the manufacturing for the double-layer conductive textile trace.

FIG. 7 shows an image of an integrated system for textile hybrid electronics (using a laser hybrid manufacturing process) in a bent state.

FIG. 8A shows an electrocardiogram (ECG) signal acquired by the integrated system for textile hybrid electronics manufactured using this process in an initial state; and

FIG. 8B shows the ECG signal acquired by the same system after being manually crumpled for 20 seconds.

FIG. 9A shows an image of the integrated system for textile hybrid electronics manufactured using this process before cleaning; and

FIG. 9B shows an image of the same system after cleaning.

FIG. 10A shows a photograph of a multifunctional wireless textile sensing patch manufactured using this process;

FIG. 10B shows a placement position of the sensing patch on a human body; and

FIG. 10C shows real-time signals displayed on a signal visualization interface when the sensing patch is worn on the human body.

### DETAILED DESCRIPTION

The technical solutions of the present disclosure are further described below in combination with specific embodiments and accompanying drawings.

According to a specific embodiment of the present disclosure, an integrated system for textile hybrid electronics via laser hybrid manufacturing specifically includes the following preparation steps:

### In S1: Manufacturing of an LIG Sensor

A PI fabric is selected as a substrate. A CO₂ laser is employed to irradiate the PI textile, converting it into LIG via a photothermal reaction to obtain a patterned LIG sensor, which is sensitive to skin temperature and strain.

The CO₂ laser is employed to irradiate connection points of the LIG sensor from the other side of the aforementioned PI fabric substrate, causing the LIG to penetrate through the thickness of the PI fabric, thereby electrically routing the LIG sensor out from the other side of the PI fabric substrate.

### In S2: Manufacturing of VIAs

LM and Cu NP are mixed using a centrifugal mixer to form an LM/Cu NP paste with good wettability. This paste is printed onto predetermined locations on two sides of the PI fabric substrate.

Air is blown onto the LM/Cu NP paste from the two sides using an air gun, causing the LM/Cu NP paste to infiltrate pores of the PI fabric, establishing vertical electrical conduction between the LM/Cu NP on the two sides of the PI fabric, thereby obtaining LM/Cu NP VIAs.

### In S3: Manufacturing of Double-Layer Conductive Textile Traces and Physiological Electrodes by Combining Laser Cutting and Transfer Printing

A laser-perforated AF is attached to an MT to obtain an AF/MT laminate. Subsequently, a tacky water-soluble SL is attached to an MT side of the AF/MT laminate, obtaining an AF/MT/SL laminate. A UV nanosecond laser is employed to cut the AF/MT/SL laminate from an AF side. During this process, a laser spot followed an outer contour of the designed conductive traces and physiological electrodes. Moreover, laser energy cut through the AF and MT while leaving the SL intact.

The AF side of the resulting AF/MT/SL laminate is attached to the aforementioned PI fabric substrate, forming a PI/AF/MT/SL stack. Subsequently, the PI/AF/MT/SL stack is immersed in water to dissolve the SL, obtaining a PI/AF/MT structure. After drying the PI/AF/MT, the excess AF/MT material outside the laser-cut contour is peeled off, thereby obtaining the conductive textile traces and physiological electrodes adhered to the PI fabric. The PI/AF/MT structure is hot-pressed to further enhance the adhesion of the AF.

The above steps are repeated on the other side of the PI fabric substrate to obtain the double-layer conductive textile traces. The PI fabric substrate electrically insulates the conductive traces on different layers, allowing conduction only at the locations of the LM/Cu NP VIAs.

### In S4: Integration of General SMDs

Following the aforementioned steps, patterned conductive textile traces and physiological electrodes are obtained on a surface of the PI fabric. A tin-based solder paste and an aluminum flux are stirred and mixed at a volume ratio of 5:1 to produce a low-melting mixed slurry capable of removing an oxide layer on surfaces of the conductive textile traces. SMDs are soldered onto the conductive textile traces using this composite slurry.

The integrated system for textile hybrid electronics manufactured via the technical solutions of the present disclosure maintains full functionality after repeated washing and mechanical crumpling, demonstrating high integration robustness between the electronic components and the textile substrate. Moreover, by implementing an independently routed dual-layer conductive circuit on a single-layer textile, the system can achieve universal circuit functions on a breathable fabric substrate, allowing for wireless monitoring of three physiological signals, such as heart rate, respiration, and ECG, without requiring additional commercial printed circuit boards (PCBs) or wired connections. Several key technical aspects of the system lie in:
(1) excessive laser energy is prevented from damaging the textile substrate by combining laser cutting with transfer printing. To ensure that excess conductive textile material can be cleanly removed along laser-cut seams, slightly higher laser energy is often required. If the transfer printing process is not used, that is, if the uncut conductive textile is first attached to the textile substrate and laser-cut, the excessive laser energy will act directly on the textile substrate, compromising its structural integrity and appearance. By adopting the transfer printing process, the conductive textile is placed over an SL rather than the textile substrate during laser cutting. Consequently, any overflow laser energy is absorbed by the SL and does not reach the textile substrate. After cutting, the conductive traces and physiological electrodes are spatially discontinuous but remain in position relative to each other due to the support of the SL. Once transferred onto the textile substrate, the SL can be dissolved in cold water under mild conditions, leaving the textile substrate and the conductive media undamaged.
(2) The use of tangential force provided by the centrifugal mixer facilitates the preparation of the LM/Cu NP paste. The intermetallic compound formed by reacting gallium-based LM with other metal nanoparticles exhibits semi-solid, paste-like physical properties, overcoming the high surface tension of common LMs such as eutectic gallium-indium (EGaIn) and gallium-indium-tin (Galinstan), thus making it easy to apply onto rough textile surfaces. Such intermetallic compounds are typically prepared by manual grinding, acid treatment, or alkali treatment. Manual grinding is inefficient and results in poor dispersion between the LM and Cu NPs. Acid treatment can cause a certain degree of damage to the Cu NPs, and the dosage is difficult to control. Alkali treatment, such as immersion in a sodium hydroxide (NaOH) solution, leaves residual NaOH in the paste, which is difficult to remove completely. In the present disclosure, the tangential force generated during centrifugation is utilized to break an oxide film on a surface of the LM, allowing for full reaction between the LM and Cu NPs during mixing without introducing additional chemical reagents. The mixing is uniform and requires only a single processing step. The resulting LM/Cu NP composite possesses sufficient fluidity to infiltrate the pores of the textile via a pneumatic method, thereby forming the LM/Cu NP VIAs.
(3) The mixed slurry of tin-based solder paste and aluminum flux is used for soldering. The outermost layer of commercially available conductive textiles is predominantly metallic nickel, whose oxide layer is difficult to remove with conventional fluxes in standard solder pastes. Aluminum flux is adopted to remove a nickel oxide layer, allowing subsequent soldering of the conductive textile with tin-based solder paste. By mixing the tin-based solder paste and aluminum flux together, the removal of the nickel oxide layer and the soldering process can be accomplished in a single step, thereby reducing the number of working procedures.

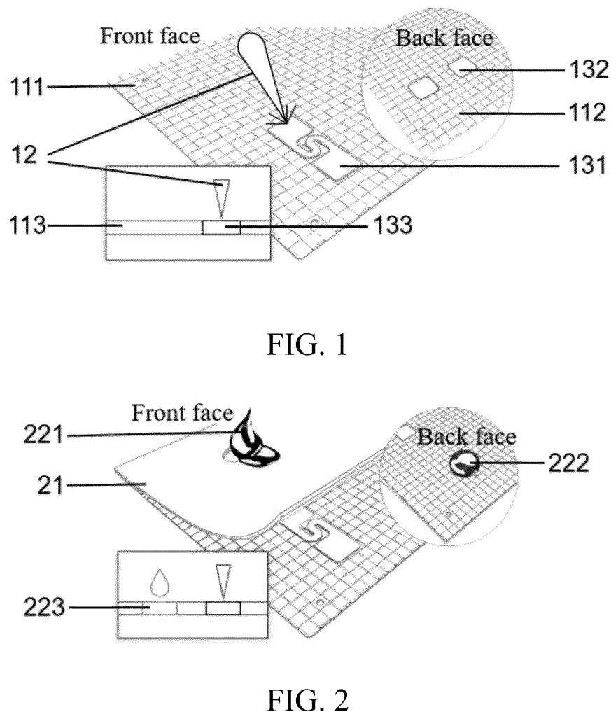

Specifically:
referring to FIG. 1, to manufacture an LIG sensor, a CO₂ laser 12 (wavelength 10.6 μm, spot radius 76 μm) is employed to process a front face 111 of a PI fabric substrate, forming a patterned LIG sensor 131 and alignment holes (diameter approximately 600 μm). After coating the LIG sensor with Ecoflex 00-50, the CO₂ laser 12 is employed to process an overlapping area on a back face 112 of the PI fabric substrate and the LIG sensor 131, creating LIG contact points 132, forming a through-fabric LIG conductive pathway 133 in a cross-section 113 of the PI fabric substrate, ultimately electrically routing the LIG sensor out from the other side 112 of the PI fabric substrate.

Referring to FIG. 2, to manufacture VIAs, LM EGaIn and Cu NPs are mixed at a mass ratio of 50:1 in a high-speed defoaming mixer to synthesize an LM/Cu NP paste. Subsequently, the LM/Cu NP paste is printed onto target locations using a polyethyleneterephthalate (PET) mask and a brush. An air gun (with a nozzle diameter of 3 mm and pressure of 200 kPa) is employed to blow air onto the LM/Cu NP paste 221 from one side. The PI fabric substrate is flipped, and the same air-blowing operation is performed on the LM/Cu NP paste 222 on the other side. An LM/Cu NP VIA 223 is formed at the location where the LM/Cu NP paste 221 and the LM/Cu NP paste 222 overlap.

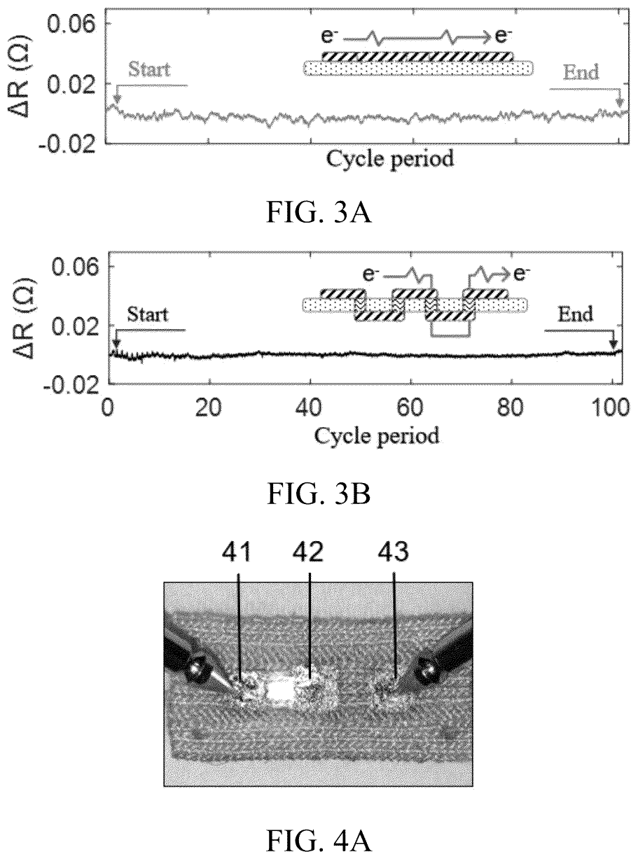

As shown in FIGS. 3A and 3B, repeated bending tests are conducted on a single-layer conductive textile trace and a double-layer conductive textile trace with four LM/Cu NP VIAs. FIG. 3A shows a resistance change of the single-layer trace under repeated bending, while FIG. 3B shows that of the double-layer trace with four LM/Cu NP VIAs. It can be observed that two types of traces exhibit similar disturbance resistance, indicating that the LM/Cu NP VIAs do not compromise the basic functionality of the textile-based circuit.

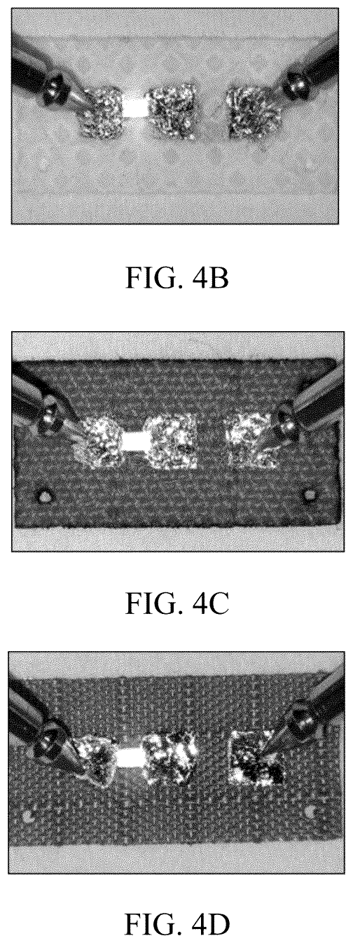

As shown in FIGS. 4A, 4B, 4C, and 4D, LEDs are connected in series with LM/Cu NP VIAs and mounted on modal knitted fabric (FIG. 4A), medical non-woven fabric (FIG. 4B), PI fabric (FIG. 4C), and taffeta woven fabric (FIG. 4D). Taking FIG. 4A as an example, an LED is soldered between an electrode 41 and an electrode 42. The electrode 42 is connected to an electrode 43 via two LM/Cu NP VIAs and a conductive trace on the back face of the fabric. When a voltage is applied between the electrode 41 and the electrode 43, the LED is illuminated. This demonstrates that the provided LM/Cu NP VIAs can meet the design requirements of complex textile circuits and achieve the transmission of physiological signals across fabric layers.

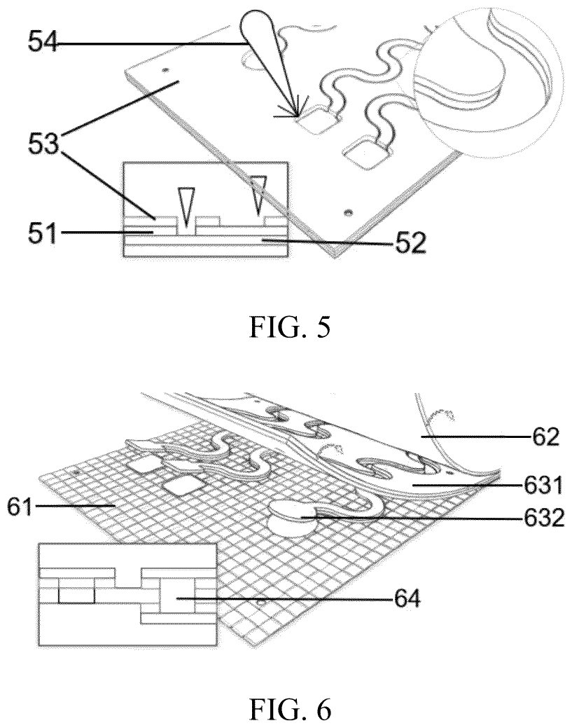

As shown in FIG. 5, to manufacture double-layer conductive textile traces, an AF 53 is laminated onto one side of an MT 51, and a tacky water-soluble SL 52 is laminated onto the other side of the MT 51. Subsequently, a UV nanosecond laser 54 (wavelength 355 nm, spot radius 16 μm) is employed to cut the MT 51 and the AF 53 into a patterned conductive circuit, while the underlying water-soluble SL 52 remains intact, providing temporary fixation for the conductive circuit. An enlarged conceptual diagram of a cut section is presented in a circular inset in an upper-right corner of FIG. 5.

As shown in FIG. 6, the aforementioned AF/MT/SL laminate is attached to the textile substrate 61, and the entire structure is immersed in water. The SL 62 softens in water and can be removed. Subsequently, the remaining AF/MT layers adhered to the textile substrate are dried, and the excess AF/MT material 631 is peeled away, leaving behind the patterned AF/MT 632, which serves as the conductive textile traces or physiological electrodes. By repeating the same process on the other side of the textile substrate 61, circuits on the front and back faces are obtained. The circuits on the front and back faces are connected by LM/Cu NP VIAs 64, thereby realizing a vertically interconnected double-layer textile circuit. The two faces of the double-layer textile circuit are hot-pressed at 80° C. to enhance the adhesion between the conductive traces, electrodes, and the textile substrate.

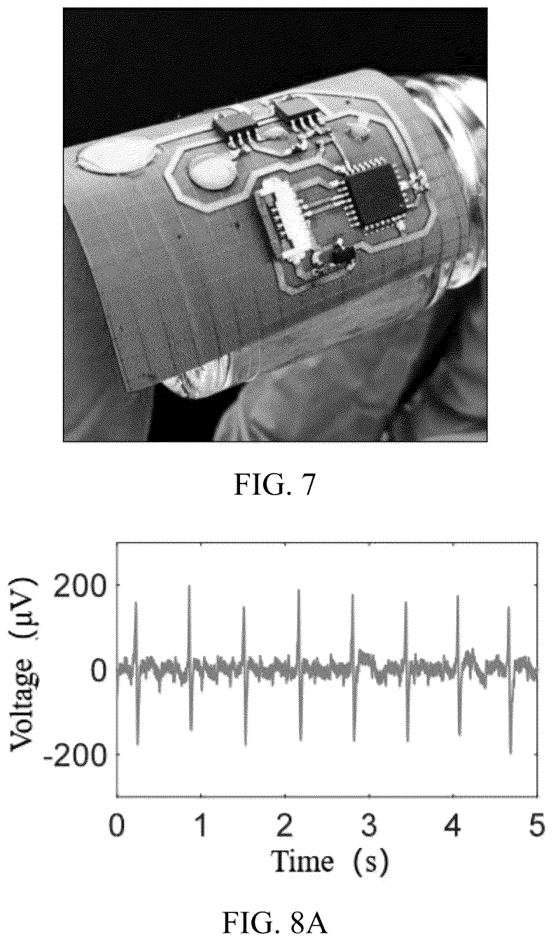

As shown in FIG. 7, to evaluate the bend resistance of the integrated system for textile hybrid electronics, the fabric is bent to a curvature radius of 25 mm. The SMDs on the fabric remain unaffected, demonstrating that the hybrid textile-electronic system possesses excellent bending durability.

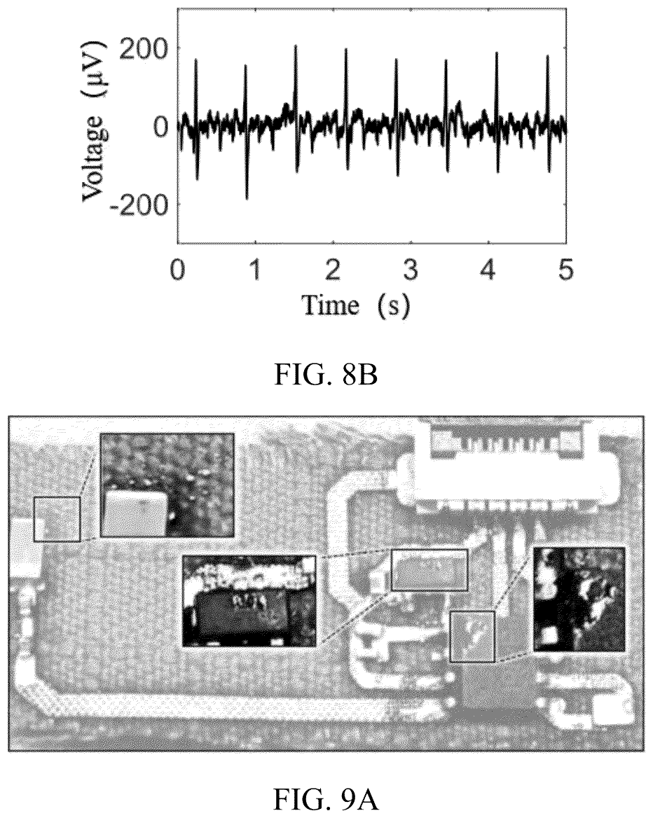

As shown in FIGS. 8A and 8B, to further verify the stability of the textile hybrid electronic system, the fabric is manually crumpled for approximately 20 seconds and used for acquiring ECG signals. FIG. 8A shows an ECG signal acquired before crumpling, while FIG. 8B shows an ECG signal acquired after crumpling. It can be observed that the ECG signals collected before and after crumpling are virtually identical, indicating that the integrated system for textile hybrid electronics exhibits considerable durability.

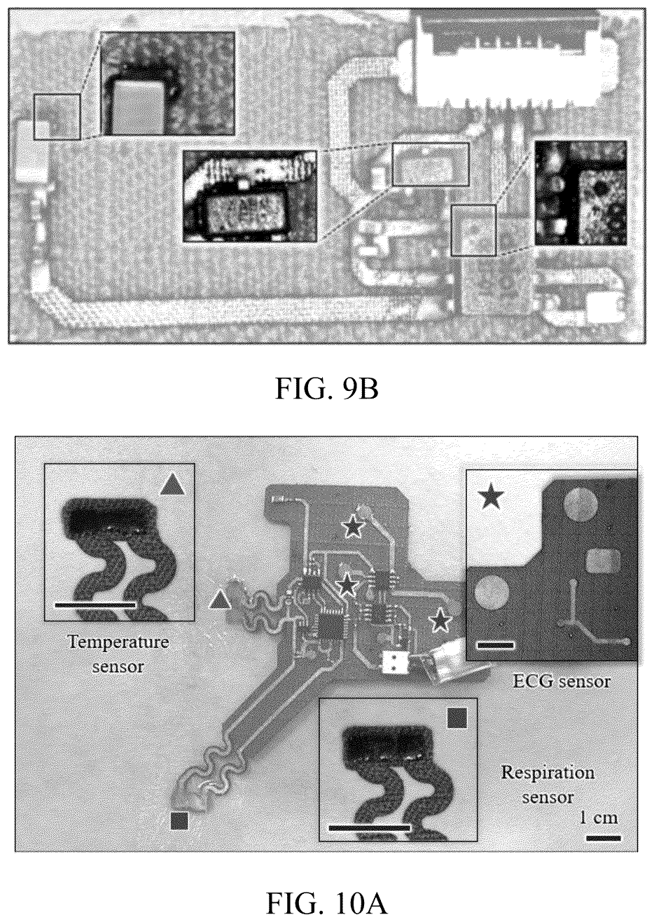

As shown in FIGS. 9A and 9B, to further evaluate the washing durability of the integrated system for textile hybrid electronics, the fabric is immersed in detergent and subjected to hand washing and ultrasonic cleaning. FIG. 9A shows a fabric image before cleaning, and FIG. 9B shows a fabric image after cleaning. It can be observed that stains and residual solder flux on the fabric are removed after cleaning, while a Bluetooth module remains capable of communicating with a mobile phone. This feature facilitates long-term maintenance of the integrated system for textile hybrid electronics.

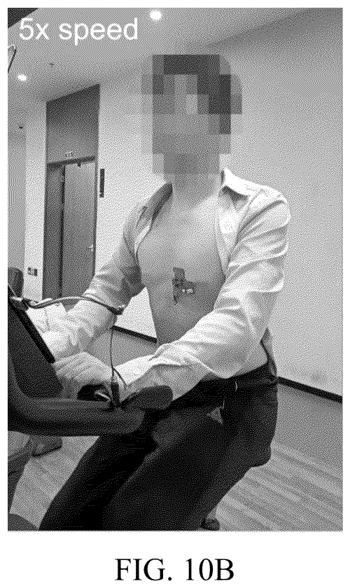

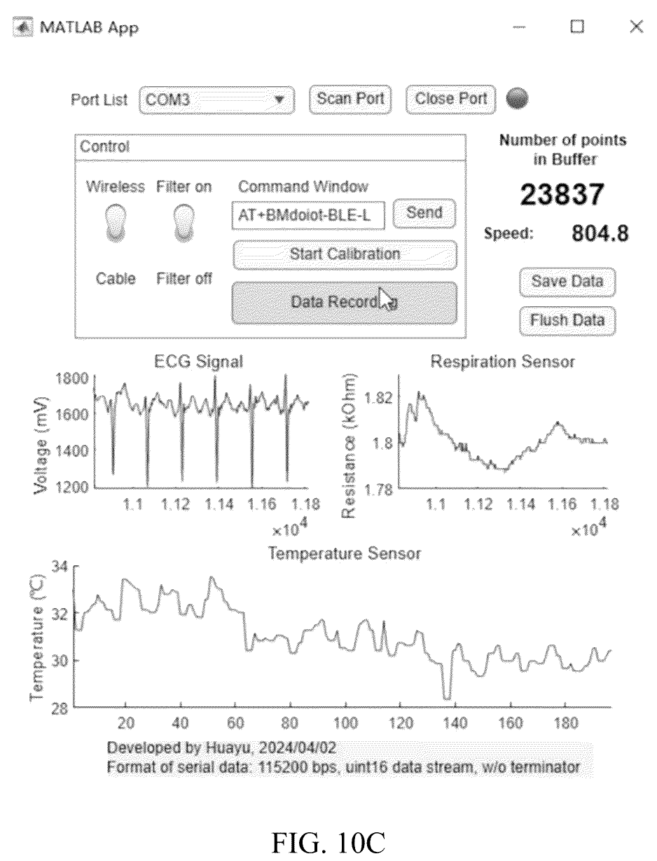

As shown in FIGS. 10A and 10B, the LIG sensor, physiological electrodes, double-layer conductive textile traces, and rigid silicon-based chips are integrated onto a single-piece textile using the laser hybrid manufacturing process. FIG. 10A shows a photograph of the multifunctional sensing, wireless integrated system for textile hybrid electronics. To validate the signal acquisition and transmission functions of this integrated system for textile hybrid electronics, the textile is placed at an appropriate position near a left chest of a test subject as illustrated in FIG. 10B, followed by an indoor cycling exercise. FIG. 10C demonstrates that during exercise, the integrated system for textile hybrid electronics can successfully acquire three types of physiological signals, such as heart rate, respiration, and ECG, and wirelessly transmit these signals to a host computer for data visualization and real-time processing.

1. An integrated system for textile hybrid electronics via laser hybrid manufacturing, comprising a textile substrate, conductive textile traces and physiological electrodes, vertical interconnect accesses (VIAs), laser-induced flexible sensors, and surface mounted devices (SMDs), wherein the textile substrate is made of one or more materials selected from the group consisting of: modal knitted fabric, medical non-woven fabric, polyimide (PI) fabric, and taffeta woven fabric; all electronic components are densely integrated on a single textile substrate and distributed on surfaces of two sides of the textile substrate, interconnected to achieve complete circuit functionality; and a preparation process specifically comprises the steps of:
S1, formation of the laser-induced flexible sensors: modifying the textile substrate or precursor materials on a surface of the textile substrate using a laser according to a pre-designed pattern, to generate the laser-induced flexible sensors on one side of the textile substrate for detecting physical and chemical signals on a human body surface; and electrically routing the laser-induced flexible sensors out from the other side of the textile substrate;
S2, formation of the VIAs: employing a pneumatic method to infiltrate conductive paste into textured structures of the textile substrate at locations on the textile substrate where VIAs are required to connect circuits on two sides of the textile substrate, to achieve electrical connectivity between front and back faces at corresponding regions of the textile substrate, with the conductive paste being an intermetallic compound formed from a liquid metal (LM)/copper nanoparticle (Cu NP) mixture, which is prepared using a centrifugal mixer;
S3, formation of the conductive textile traces and physiological electrodes: cutting a conductive textile using a laser according to a pre-designed pattern to form the conductive traces and physiological electrodes; and transferring the conductive traces and electrodes onto the two sides of the textile substrate using a transfer-printing process, causing the conductive traces and electrodes on the two sides to be electrically interconnected through the VIAs; and
S4, integration of the SMDs: soldering the SMDs onto the conductive textile traces using a low-melting solder paste, forming a complete circuit.

2. The integrated system for textile hybrid electronics via laser hybrid manufacturing according to claim 1, wherein the formation of the laser-induced flexible sensors is specifically achieved as follows: using a photothermal effect of either a carbon dioxide (CO₂) laser or a 532 nm continuous-wave green laser, the textile substrate or precursor materials on a surface of the textile substrate are processed according to a pre-designed pattern, and reactions comprising carbonization, phase separation, or reduction-sintering are induced, generating the laser-induced flexible sensors on one side of the textile substrate for detecting physical and chemical signals on a human body surface; and the laser-induced flexible sensors are routed electrically out from the other side of the textile substrate.

3. The integrated system for textile hybrid electronics via laser hybrid manufacturing according to claim 1, wherein the conductive textile traces and physiological electrodes are prepared by combining laser cutting of conductive textiles with a transfer printing process: during laser cutting, a water-soluble sacrificial layer (SL) is utilized to temporarily secure the conductive textile traces and physiological electrodes; after cutting is completed, the conductive textile traces and physiological electrodes are transferred onto the textile substrate; and the SL is dissolved in water to prevent laser-induced damage to the textile substrate.

4. The integrated system for textile hybrid electronics via laser hybrid manufacturing according to claim 3, wherein a method for preparing the conductive textile traces and physiological electrodes specifically comprises: laminating an adhesive film (AF) onto one side of a metalized textile (MT) and laminating a tacky water-soluble SL onto the other side of the MT to form an AF/MT/SL laminate; cutting AF and MT layers into patterned conductive circuits using an ultraviolet (UV) nanosecond laser while keeping the SL intact; adhering the entire laminate to the textile substrate with an AF face facing down; immersing the textile substrate in water to soften the SL, followed by removing the SL; drying the remaining AF/MT layers on the textile substrate; and removing excess AF/MT material outside the patterned conductive circuits to form the conductive textile traces or physiological electrodes.

5. The integrated system for textile hybrid electronics via laser hybrid manufacturing according to claim 1, wherein the low-melting solder paste is a mixed slurry of tin-based solder paste and aluminum flux.
